(19) United States
(12) Patent Application Publication
(10) Pub. No.: US 2015/0150712 A1
(43) Pub. Date: Jun. 4, 2015

(54) CURABLE ORTHOPEDIC SPLINT

(71) Applicant: WOOSAM MEDICAL CO., LTD, Jongno-gu, Seoul (KR)

(72) Inventor: Tae-Hee Huh, Seoul (KR)

(73) Assignee: WOOSAM Medical Co., Ltd., Jongno-gu, Seoul (KR)

(21) Appl. No.: 14/400,775

(22) PCT Filed: Jan. 22, 2013
(86) PCT No.: PCT/KR2013/000507

§ 371 Date: Nov. 12, 2014

(30) Foreign Application Priority Data
Jun. 25, 2012 (KR) 10-2012-0067880
Dec. 4, 2012 (KR) 10-2012-0139802

(51) Int. Cl. A61F 5/058

(52) U.S. Cl. CPC A61F 5/058

(57) ABSTRACT

Provided is a curable orthopedic splint. The curable orthopedic splint includes: an inner shell contacting a bone fracture site, an outer shell bonded to the inner shell, a curable core accommodated between the inner shell and the outer shell, the curable core being cured to match a shape of the bone fracture site, and a fixing holder contacting a surface of the outer shell to surrounding the curable core and the bone fracture site, the fixing holder having both ends that are detachably bonded to each other or detachably bonded to the surface of the outer shell so as to closely attach and fix the curable core to the bone fracture site.

### FIELD OF THE INVENTION

The present invention relates to a curable splint for orthopedics, and more particularly, to a curable orthopedic splint, which is helpful in treating a bone fracture site through fixing of the fracture site so as not to move, when a body part such as an arm, a leg, etc., is broken or sprained.

### BACKGROUND OF THE INVENTION

In general, when an arm or a leg is broken or sprained, non-surgical treatment is performed such that the injured fracture site is fixed by using an orthopedic splint so as not to move.

Conventionally, a fixing method using a plaster-applied bandage wrapped around the injured area has been widely used as an orthopedic splint. However, in this treatment, a shrinkage phenomenon occurs while plaster is cured, and thus the injured area cannot be effectively supported. In addition, it takes a relatively long time to wrap the bandage around the injured area. Also, due to a lack of air permeability, a problem arises such that a lot of sweat comes out to cause itching when used for a long time. Particularly, when the injured area is healed up with the curing of the plaster completed after the treatment, a separate cutter should be used to remove the plaster. Here, a problem arises such as skin hurt during cutting process, etc.

In order to solve the problems of the orthopedic splint using a plaster bandage, a splint has been widely used in recent years, which has improved formability by using an elastic fiber by using one of polyester knitted fabric, glass fiber knitted fabric, non-woven fabric, or etc, which is impregnated with a curable resin such as water-curable polyurethane. Such splints are circulated in a packaged state, and the packaging material is removed in use so that the inner elastic fabric may be quickly and naturally cured at room temperature, thereby allowing an enhanced formability.

When a splint using a curable resin is used in the treatment, firstly, air-tight vacuum packing of the splint is unpacked, and the splint is immersed into water so that the water is sufficiently penetrated into a curable core in the splint. Then, the splint is squeezed dry and wiped with a towel and then closely attached to match the shape of the injured area. Next, the whole injured area contacting the splint is wrapped with a high elastic bandage, or the splint which is contained in a packaging sheet and impregnated with the curable resin, is taken out at room temperature and wrapped around the injured area. Then, the splint is formed to meet the condition of the injured area during the gradual curing process. As three to four minutes elapses after the treatment, the curable core is hardly cured, and the injured area is firmly fixed so as not to move. Thus, the treatment of the splint is completed.

FIG. 1 is a perspective view of a curable orthopedic splint according to a related art, and FIG. 2 is a perspective view illustrating a state in which the curable orthopedic splint is wrapped with a bandage according to the related art.

A curable orthopedic splint 10 according to a related art includes an inner shell 11 that is in contact with a bone fracture area and made of hydrophobic non-woven fabric, a hydrophilic outer shell 12 connected to the inner shell 11, and a curable core 13 built between the inner shell 11 and the outer shell 12. The curable core 13 is made of a material such as a water-curable polyurethane resin.

An application method of the curable orthopedic splint 10 according to the related art is as follows. Firstly, air-tight vacuum packing of the splint 10 is unpacked, and the splint 10 is immersed into water so that the water is sufficiently penetrated into the curable core 13 inside the splint 10. Then, the splint 10 is squeezed dry and wiped with a towel, and then the splint 10 is closely attached to match the shape of the injured area. Next, the whole injured area contacting the splint 10 is wrapped with a high elastic bandage 20, and thus the splint 10 is used in fixed state.

Since the inner shell 11 directly contacts a skin, a soft and volumetric non-woven fabric having a depth of about 3 mm to about 4 mm may be used. Also, since the outer shell 12 needs high elongation, a non-woven fabric such as a stretch lace may be used. This is well-known in the art. The curable core 13 is coated with the curable resin to absorb water, thereby being cured.

However, according to the above-mentioned curable orthopedic splint according to the related art, there are problems as follows.

First, since the whole injured area is fixed by wrapping a high elastic bandage 20 around the injured area while a bone fracture site is kept to be in the state where the fracture site is closely attached to the inner shell 11 to match a shape of the injured area, it is very uncomfortable to wear and take off the splint to/from the bone fracture site. Particularly, a fracture patient has much difficulty in self-wearing and self-taking off.

Secondly, since the whole injured area is wrapped around by the high elastic bandage 20, there is a problem in that a lot of sweat comes out, thereby causing itching due to a lack of air permeability.

### SUMMARY OF THE INVENTION

Accordingly, the present invention is directed to provide a curable orthopedic splint capable of being easily wore and taken off to/from a bone fracture site that substantially obviates one or more problems due to limitations and disadvantages of the related art.

Also, the present invention is directed to provide a curable orthopedic splint that is capable of providing air permeability.

According to an aspect of the present invention, there is provided a curable orthopedic splint including: an inner shell contacting a bone fracture site; an outer shell bonded to the inner shell; a curable core accommodated between the inner shell and the outer shell, the curable core being cured to match a shape of the bone fracture site; and a fixing holder contacting a surface of the outer shell to surrounding the curable core and the bone fracture site, the fixing holder having both ends that are detachably bonded to each other or detachably bonded to the surface of the outer shell so as to closely attach and fix the curable core to the bone fracture site.

Bonding parts, which are detachably bonded to each other, may be disposed on both ends of the fixing holder.

The curable orthopedic splint may further include a fixing member for fixing the fixing holder to the outer shell.

The fixing member may be bonded to the outer shell, and through-holes through which the fixing holder passes may be defined between the fixing member and the outer shell.

The plurality of through-holes may be defined between the fixing member and the outer shell so that the fixing holder passes through all of the through-holes, or the fixing member selectively passes through a portion of the through-holes to freely adjust the number of the fixing holders and fixing positions of the fixing holders.

A slit into which a bonding part between the through-holes is inserted may be defined in the fixing holder, and a connection part for inserting the bonding part into the slit may be disposed on the fixing holder.

The fixing member may include: a first fixing part disposed on entire outer surface or a portion of the outer surface; and a second fixing part disposed on the fixing holder and detachably bonded to the first fixing part.

The first fixing part may have a length in which the plurality of fixing holders are attached to the first fixing part, the fixing holder may be freely changed in bonding position on the first fixing part, and the number of fixing holders attached to the fixing part may be freely adjusted.

The Velcro type fixing member may be disposed on the fixing holder and detachably bonded to the surface of the outer shell.

### BRIEF DESCRIPTION OF THE DRAWINGS

FIG. 1 is a perspective view of a curable orthopedic splint according to a related art.

FIG. 2 is a perspective view illustrating a state in which the curable orthopedic splint wrapped with a bandage according to the related art.

FIG. 3 is a perspective view of a curable orthopedic splint according to a first embodiment of the present invention.

FIG. 4 is a cross-sectional view taken along line A-A of FIG. 3.

FIG. 5 is a perspective view illustrating a state in use of the curable orthopedic splint according to the first embodiment of the present invention.

FIG. 6 is a perspective view of a curable orthopedic splint according to a second embodiment of the present invention.

FIG. 7 is a perspective view of a curable orthopedic splint according to a third embodiment of the present invention.

FIG. 8 is a perspective view of a curable orthopedic splint according to a fourth embodiment of the present invention.

FIG. 9 is a perspective view illustrating a state in use of the curable orthopedic splint according to the fourth embodiment of the present invention.

FIG. 10 is a perspective view of a curable orthopedic splint according to a fifth embodiment of the present invention.

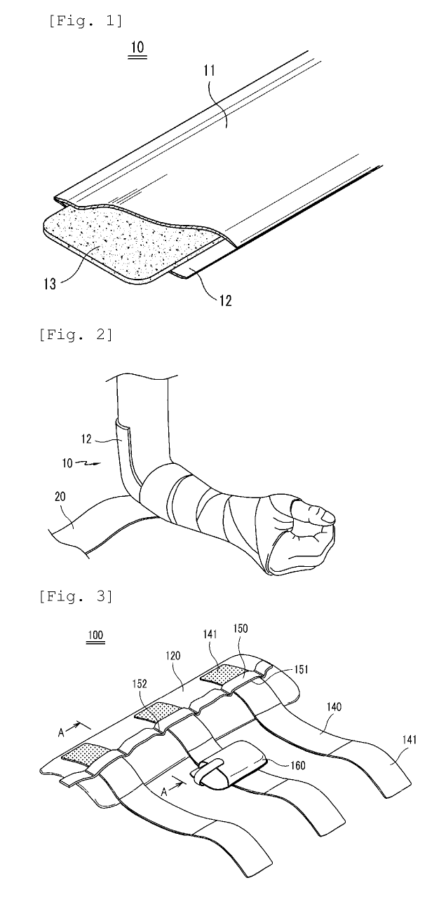

### DETAILED DESCRIPTION

Reference will now be made in detail to the preferred embodiments of the present invention, examples of which are illustrated in the accompanying drawings. Wherever possible, the same reference numbers will be used throughout the drawings to refer to the same or like parts.

Hereinafter, curable orthopedic splint according to an embodiment of the present invention will be described in detail with reference to the accompanying drawings.

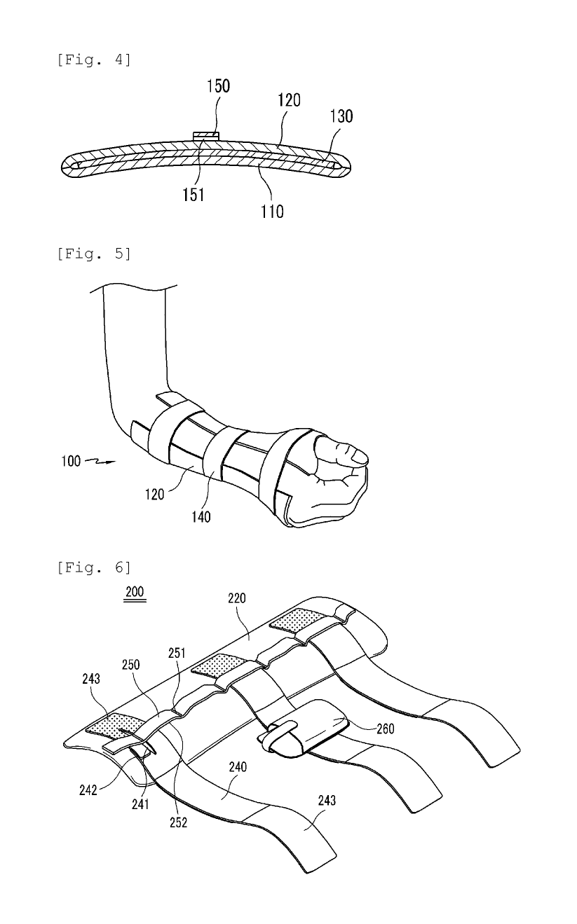

FIG. 3 is a perspective view of a curable orthopedic splint according to a first embodiment of the present invention, FIG. 4 is a cross-sectional view taken along line A-A of FIG. 3, and FIG. 5 is a perspective view illustrating a state in use of the curable orthopedic splint according to the first embodiment of the present invention.

The curable orthopedic splint 100 according to the present embodiment includes an inner shell 110, an outer shell 120, a curable core 130, a fixing holder 140, a fixing member 150 and a pocket 160.

The inner shell 110 contacting a bone fracture site is formed of a hydrophobic material. Thus, although the curable core 130 is cured to match the shape of the bone fracture site through contacting with the water, water may not contact the bone fracture site because the water is blocked by the inner shell 110.

Both ends of the outer shell 120 are bonded to both ends of the outer shell 120, and the outer shell 120 is formed of a hydrophilic material. Thus, when the curable core 130 is cured through contacting with the water, the water may be quickly penetrated and transferred to the curable core 130.

Both ends of the inner shell 110 and the outer shell 120 are bonded by a laser fusion method, and a space is defined between the inner shell 110 and the outer shell 120 so that the curable core 130 is inserted into the space. The hydrophobic material of the inner shell 110 and the hydrophilic material of the outer shell 120 are well known in the art. Thus, detailed descriptions thereof will be omitted.

The curable core 130 is embedded between the inner shell 110 and the outer shell 120 and is cured to match the shape of the bone fracture site through contacting with the water. That is, water is supplied to the curable core 130 through the outer shell 120 so as to contact the curable core 130, and then the curable core 130 is transformed to match the shape of the bone fracture site. Thereafter, when a predetermined time elapses, the curable core 130 is cured to firmly support the bone fracture site.

The curable core 130 is circulated in a packaged state by using an elastic fiber which is one of polyester knitted fabric, glass fiber knitted fabric, non-woven fabric, or etc, impregnated with a curable resin such as water-curable polyurethane. When the curable core 130 is used, the package of the curable core 130 is removed so as to contact the water and to be cured.

For reference, the curable core 130 has a width of about 2 inches to about 6 inches and a length of about 10 inches to 45 inches in general. However, the length becomes longer up to about 4 m to about 5 m in some cases. Various materials, for example, a glass fiber fabric and a non-woven fabric are being developed and used as a material of the curable core 130. In the case of the glass fiber fabric, a multi-ply, typically, an eight-ply glass fiber fabric may be stacked and used as a material for an intermediate layer. The glass fiber material has merits in good properties such as strength, air permeability, and elasticity. On the other hand, the glass fiber material is relatively expensive and heavy, and also has danger when cut. The splint should be used after being properly cut according to a position and size of a bone fracture site. However, when the glass fiber fabric is cut, every single strand of glass fiber may protrude, and thus the protruding glass fiber may hurt a skin. The non-woven fabric material may be a fabric material that has been developed to improve the shortcomings of the glass fiber material. The non-woven fabric that is developed as a material of the intermediate layer of the splint has merits in lightness, low price, and no danger in cutting.

For the orthopedic splint according to the present invention, the curable core 130 may be manufactured by using materials such as polyester and non-woven fabric as well as glass fiber fabric. In the case of the glass fiber fabric and polyester material, the curable core 130 may be manufactured to have the seven-ply, eight-ply, or less. In the case of the non-woven fabric material, the curable core 130 may be manufactured to have a multi-ply as well as a single-ply.

The fixing holder 140 is fixed to a surface of the outer shell 120 to surround the curable core 130 and the bone fracture site, thereby allowing the curable core 130 to be closely attached to the bone fracture site. Bonding parts 141 which are detachably bonded to each other are disposed at both ends of the fixing holder 140. The bonding parts 141 may preferably be a Velcro type. However, the type of the bonding parts 141 is not limited to this, but allowed to be a button type. The bonding parts 141 are formed to have a length that is changeable according to the size of the bone fracture site.

The fixing member 150 is disposed to fix the fixing holder 140 to the outer shell 120 and is fused and bonded to the outer surface of the outer shell 120. Thus, a plurality of through-holes 151 through which the fixing holder 140 passes are defined to be spaced apart from each other between the outer shell 120 and the fixing member 150. A plurality of fusion bonding parts 152 are formed on the fixing member 150, with a predetermined distance from each other and each fusion bonding part is attached to a surface of the outer shell 120 by a method, such as laser fusion, heat fusion, etc. The fusion bonding parts 152 are fused and bonded to outer shell 120, and thus the through-holes 151 are naturally defined.

The fixing holder 140 may freely move in the though-holes 151 defined by the fixing member 150. The fixing member 140 may be disposed as many as the number of the through-holes 151 and be inserted into and fixed to all the through-holes 151 or to only a portion of the through-holes 151. When the fixing holder 140 is inserted and fixed to only a portion of the through-holes 151, the fixed positions thereof may be freely changeable.

The pocket 160 may be bonded to the fixing holder 140 by the Velcro type member.

A process of using a curable orthopedic splint including the above-described constitutions according to a first embodiment of the present invention will now be described.

After a packing sheet is removed, a curable orthopedic splint according to the present invention is immersed into water so that the water is sufficiently penetrated into a curable core 130 inside a splint 10. Then, the splint 10 is squeezed dry and wiped with a towel, and then the splint 10 is closely attached to a bone fracture site through an inner shell 110 and transformed to match the shape of the injured area.

After about three to about four minutes, the curable core 130 is cured to match the shape of the bone fracture site.

When the curable core 130 is cured, a fixing holder 140 is inserted into through-holes 151 of a fixing member 150, and the bone fracture site is wrapped by the fixing holder 140. Then, both ends of the fixing holder 140 are pulled to be closely attached to the bone fracture site, and bonding parts 141 formed at both ends of the fixing holder 140 are bonded to each other.

The number of the fixing holders and positions of the fixing holders 140 may be different according to the fracture degree and a range of the bone fracture site.

When the curable orthopedic splint 100 according to the present invention needs to be taken off (to be untied) for sleeping or according to needs, the splint 10 may be easily taken off by only separating the bonding parts 141 of the fixing holder 140 from each other.

In the case where the fixing holders 140 are inserted into only a portion of the through-holes 151, and also, even in the case where the fixing holders 140 are inserted into all the through-holes, air permeability of the bone fracture site may be effectively maintained by forming a gap between the fixing holders 140 through fusion bonding parts 152. Thus, itching or offensive smell generated by excessive sweat may be prevented even after a long time.

According to the curable orthopedic splint 100 according to the present invention including the above-described constitutions, the splint 100 may be easily applied to a bone fracture site in such a way that the fixing holders 140 are inserted into the through-holes 151 of the fixing member 150, and the bone fracture site is surrounded by the fixing holders 140, and then the bonding parts 141 formed on both ends of the fixing holders 140 are bonded to each other. Also, the curable orthopedic splint 100 according to the present invention may be easily taken off by only separating the bonding parts 141 from each other.

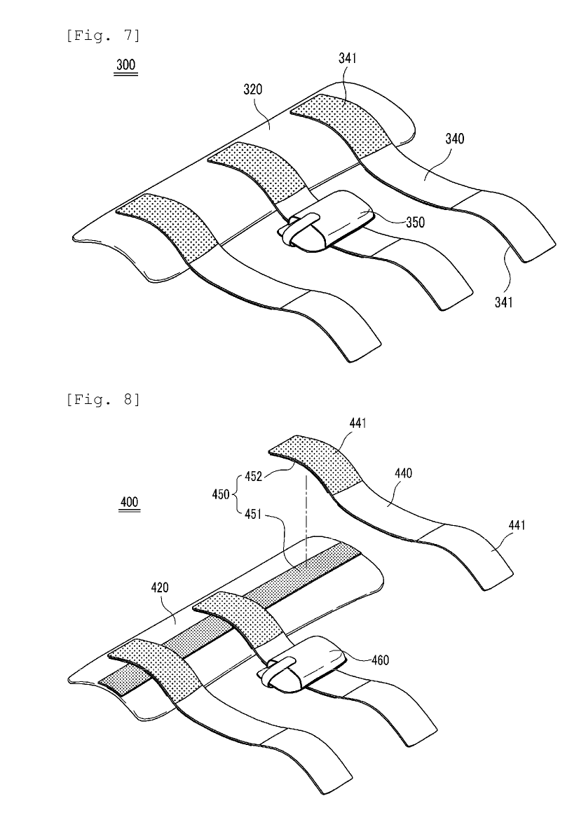

FIG. 6 is a perspective view of a curable orthopedic splint according to a second embodiment of the present invention.

A curable orthopedic splint 200 according to the present embodiment includes an inner shell (not shown), an outer shell 220, a curable core (not shown), a fixing holder 240, a fixing member 250, and a pocket 260. Here, the inner shell, the outer shell 220, the curable core (not shown), the fixing member 250, and the pocket 260 are the same as the first embodiment and therefore descriptions thereof will be omitted.

The curable orthopedic splint 200 according to the present embodiment has a slit 241 which is formed in the fixing holders 240, and into which a fusion bonding part 251 of the fixing member 250 are inserted. The fixing holder 240 may be fixed to the fixing member 250 by using the slit 241.

Connection parts 242 for inserting the fusion bonding part 251 into the slits 241 are disposed at a side of the slit 241. That is, a connected state of each of the connection part 242 is in a disconnected state, and one end of the fixing holder 240 is inserted into a through-hole 252. Then, the fusion bonding part 251 is inserted into the slit 241 through the connection part 242, and then the connection parts 242 are connected to each other. Thus, the fixing holder 240 is fixed to the fusion bonding parts 251.

Although the connection part 242 may preferably be formed to be a Velcro type, the type of the connection part 242 is not limited to this, but allowed to be a button type.

Hereinafter, since the state of using the curable orthopedic splint 200 according to the present embodiment is the same as the first embodiment except for a difference in the process of fixing the fixing holder 240 to the fixing member 240, descriptions thereof will be omitted. Non-described reference numeral 243 represents connection parts which are disposed at both ends of the fixing holder 240 and are connected to each other.

FIG. 7 is a perspective view of a curable orthopedic splint according to a third embodiment of the present invention.

A curable orthopedic splint 300 according to the present embodiment includes an inner shell (not shown), an outer shell 320, a curable core (not shown), a fixing holder 340, and a pocket 350. Here, since the inner shell, the outer shell 320, the curable core, and the pocket 350 are the same as the first embodiment, and descriptions thereof will be omitted.

In the curable orthopedic splint 300 according to the present embodiment, the fixing holder 340 may be fused and bonded to a surface of the outer shell 320. Here, a plurality of the fixing holders 340 are disposed and bonded to be spaced apart from each other on the surface of the outer shell 320. Each of Velcro type or button type bonding parts 341, which may be detachably bonded to each other, is disposed at both ends of the fixing holder 340.

In the curable orthopedic splint 200 according to the present embodiment, it is impossible to change the positions of the fixing holders 340. However, it may be possible to easily wear and take off the fixing holder 340 to/from a bone fracture site by using the bonding parts 341 disposed at both ends of the fixing holder 340.

FIG. 8 is a perspective view of a curable orthopedic splint according to a fourth embodiment of the present invention, and FIG. 9 is a perspective view of a state in use of a curable orthopedic splint according to a fourth embodiment of the present invention.

A curable orthopedic splint 400 according to the present embodiment includes an inner shell (not shown), an outer shell 420, a curable core (not shown), a fixing holder 440, a fixing member 450, and a pocket 460. Here, since the inner shell, the outer shell 420, the curable core, the fixing holder 440, and a pocket 460 are the same as the first to third embodiment, descriptions thereof will be omitted.

The fixing member 450 is disposed to fix the fixing holder 440 to the outer shell 420 and includes a first fixing part 451 and a second fixing part 452.

The first fixing part 451 is formed in a Velcro type and elongated over an outer surface of the outer shell 420. In the present embodiment, although the first fixing part 451 is fixed to the outer shell 420 by sewing, the fixing method is not limited thereto. Thus, the first fixing part 451 may be fused and bonded to a surface of the outer shell 420 in a manner such as laser fusion, heat fusion, etc.

The second fixing part 452 is formed in a Velcro type and disposed on the fixing holder 440. The second fixing part 452 may be bonded to the fixing holder 440 by sewing or fusion, like the first fixing part 451, and also by various methods such as a release tape, etc.

The first and second fixing parts 451 and 452 are detachably bonded to each other. Since the width of the second fixing part 452 is shorter than the length of the first fixing part 451, a space is provided to fix the plurality of fixing holders 440 to the first fixing part 451. Thus, the fixing positions of the fixing holders 440 may be freely changed on the first fixing part 451, and also the number of the fixing holders 440 fixed on the first fixing part 451 may be freely adjusted.

Hereinafter, since the state of using the curable orthopedic splint according to the present embodiment is the same as the first embodiment except for a difference in the process of fixing the fixing holder 440 to the fixing member 450, descriptions thereof will be omitted. Non-described reference numeral 441 represents connection parts which are disposed at both ends of the fixing holder 440 and are detachably connected to each other.

In the present embodiment, although the first fixing part 451 fixed on the outer shell 420 is only disposed on a portion of the outer shell 420, the present invention is not limited thereto, and thus the first fixing part 451 may be disposed to entirely surround the outer shell 420. Here, the first fixing part 451 may be bonded to the outer shell 420 by various methods such as sewing, fusion, etc. Also, the first fixing part 451 is preferably formed of a hydrophilic material so that water is readily penetrated into the outer shell 420.

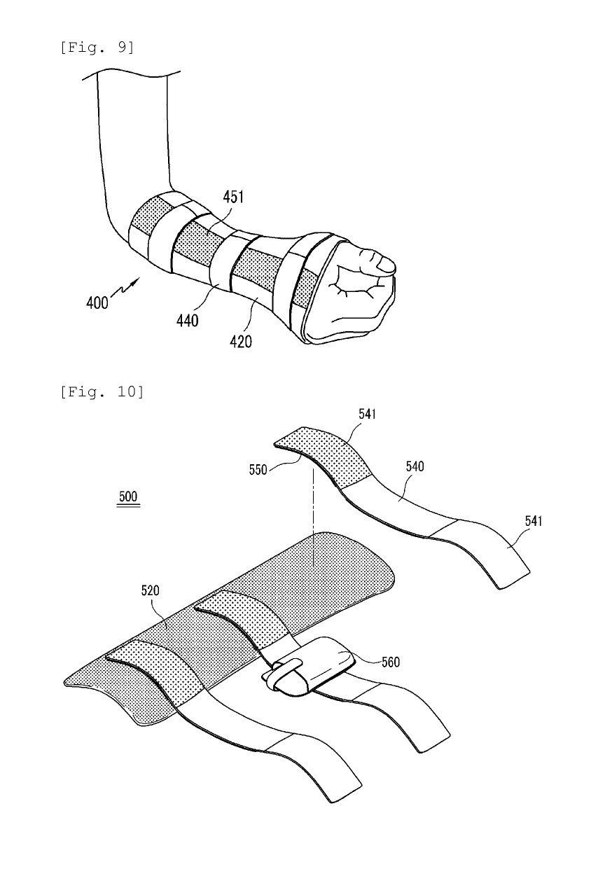

FIG. 10 is a perspective view of a curable orthopedic splint according to a fifth embodiment of the present invention.

A curable orthopedic splint 500 according to the present embodiment includes an inner shell (not shown), an outer shell 520, a curable core (not shown), a fixing holder 540, a fixing member 550, and a pocket 560. Here, the inner shell, the curable core, the fixing holder 540, and a pocket 560 are the same as the first to fourth embodiment, and therefore detailed descriptions thereof will be omitted.

The outer shell 520 is formed of a hydrophilic material, which may be detachably bonded to a Velcro, so that the fixing member 550 having the Velcro shape disposed on the fixing holder 540 may be detachably bonded to the outer shell 520.

According to the curable orthopedic splint 500 of the present embodiment, a fixing position of the fixing holder 540 is not limited to a specific portion of the outer shell 420, but allowed to be the entire outer shell 420. Thus, a user may freely fix the fixing holder 540 to a desired position on the outer shell 520.

In addition, Velcro type bonding parts that are bonded to each other are disposed on both ends of the fixing holder 540.

According to the curable orthopedic splint of the embodiments of the present invention, there may be effects as follows.

Firstly, the curable core is fixed to the bone fracture site by using teh fixing holder which surrounds the bone fracture site and of which both ends are detachably bonded to each other, so that the splint may be easily worn and taken off, thereby being convenient in use.

Secondly, the splint is constituted so that the number of the fixing holders may be freely adjusted according to the range and degree of the bone fracture site and positions of the fixing holders may be freely changed, and thus convenience in use may be more enhanced.

Thirdly, the gap between the fixing holders provides the air permeability to the bone fracture site, and thus the problems such the itching or the generation of the bad smell may be prevented.

As described above, although exemplary embodiments of the present invention has been shown and described, the present invention is not limited to a specific embodiment, and it will be apparent to those skilled in the art that various modifications and variations can be made in the present invention. Thus, it is intended that the present invention covers the modifications and variations of this invention provided they come within the scope of the appended claims and their equivalents. For example, in the present embodiments, although both ends of the fixing holder are detachably bonded to each other by bonding parts, and the fixing holder is fixed to the outer shell by using the fixing member at a variable position, the present invention is not limited thereto, and thus the fixing holder may be directly fixed to the outer shell at a variable position in a state where both ends of the fixing holder are not bonded to each other.

What is claimed is:

1. A curable orthopedic splint comprising:
an inner shell contacting a bone fracture site;
an outer shell bonded to the inner shell;
a curable core accommodated between the inner shell and the outer shell, the curable core being cured to match a shape of the bone fracture site; and
a fixing holder contacting a surface of the outer shell to surrounding the curable core and the bone fracture site, the fixing holder having both ends that are detachably bonded to each other or detachably bonded to the surface of the outer shell so as to closely attach and fix the curable core to the bone fracture site.

2. The curable orthopedic splint of claim 1, wherein bonding parts, which are detachably bonded to each other, are disposed on both ends of the fixing holder.

3. The curable orthopedic splint of claim 2, further comprising a fixing member for fixing the fixing holder to the outer shell.

4. The curable orthopedic splint of claim 3, wherein the fixing member is bonded to the outer shell, and through-holes through which the fixing holder passes are defined between the fixing member and the outer shell.

5. The curable orthopedic splint of claim 4, wherein the plurality of through-holes are defined between the fixing member and the outer shell so that the fixing holder passes through all of the through-holes, or the fixing member selectively passes through a portion of the through-holes to freely adjust the number of the fixing holders and fixing positions of the fixing holders.

6. The curable orthopedic splint of claim 5, wherein a slit into which a bonding part between the through-holes is inserted is defined in the fixing holder, and a connection part for inserting the bonding part into the slit is disposed on the fixing holder.

7. The curable orthopedic splint of claim 3, wherein the fixing member comprises:
a first fixing part disposed on entire outer surface or a portion of the outer surface; and
a second fixing part disposed on the fixing holder and detachably bonded to the first fixing part.

8. The curable orthopedic splint of claim 7, wherein the first fixing part has a length in which the plurality of fixing holders are attached to the first fixing part, the fixing holder is freely changed in bonding position on the first fixing part, and the number of fixing holders attached to the fixing part is freely adjusted.

9. The curable orthopedic splint of claim 3, wherein the Velcro type fixing member is disposed on the fixing holder and detachably bonded to the surface of the outer shell, a surface of the outer shell has a length in which the plurality of fixing holders are attached the outer shell, the fixing holders are freely changeable in bonding position on the surface of the outer shell, and the number of fixing holders are freely adjustable.
